(19) United States
(12) Patent Application Publication
(10) Pub. No.: US 2019/0221295 A1
(43) Pub. Date: Jul. 18, 2019

(54) Remote Medical Analysis Systems And Methods

(71) Applicant: Vet24seven Inc., Mountain View, CA (US)

(72) Inventors: Calbert Lai, Sunnyvale, CA (US); Pamela Seavey, Sunnyvale, CA (US); Edward Blach, Monument, CO (US); Annette Puskarich, Palo Alto, CA (US); Cherice Roth, Boring, OR (US); Tammie Pearce, Tucson, AZ (US); Ross Wright, Mesquite, TX (US)

(21) Appl. No.: 16/247,369

(22) Filed: Jan. 14, 2019

Related U.S. Application Data
(60) Provisional application No. 62/616,943, filed on Jan. 12, 2018.

(51) Int. Cl. G16H 10/60; G16H 50/20; G16H 40/20

(52) U.S. Cl. CPC G16H 10/60; G16H 70/00; G16H 40/20; G16H 50/20

(57) ABSTRACT

A method for providing veterinary services includes receiving a request for veterinary services for a pet through a communication network that supports audio communications. A diagnosis of pet medical condition can be provided based at least in part on information received through an automated veterinarian service manager. In some embodiments veterinarian service manager can direct a user to a veterinarian based on diagnosis of pet condition.

### RELATED APPLICATIONS

This application claims the benefit of U.S. Provisional Application Ser. No. 62/616,943, filed Jan. 12, 2018, which is hereby incorporated herein by reference in its entirety for all purposes.

### FIELD OF THE INVENTION

This invention relates generally to the field of animal services, including pet related diagnosis and veterinary consultations.

### BACKGROUND

Pet owners can benefit from availability of veterinary information and assistance for pets that require medical attention. An effective and low cost system for simple and fast access to a qualified veterinarian or animal professional for evaluation and treatment is needed.

### SUMMARY

In one described embodiment a method for providing veterinary services includes the steps of receiving a request for veterinary services for a pet through a communication network that supports audio communications. A diagnosis of pet medical condition can be provided based at least in part on information received through an automated veterinarian service manager. In some embodiments a veterinarian service manager can direct a user to a veterinarian based on diagnosis of pet condition.

In one embodiment the veterinarian service manager is connected to a training system able to provide data related to pet related medical conditions and treatments.

In one embodiment the veterinarian service manager is connected to a database able to store and provide various information related to pet related medical conditions and treatments.

In one embodiment the veterinarian service manager is connected to a database able to store and provide various information related to a pet owner, pet, or veterinarian.

In one embodiment the veterinarian service manager further comprises an artificial intelligence-based system to determine a severity of pet medical condition.

In one embodiment a knowledge graph database can be used to store diagnostic veterinary knowledge and provide it to a veterinary diagnostic engine able to provide diagnosis of pet medical condition.

In one embodiment a language processor can be used to handle ambiguous spoken inputs and provide them to a veterinary diagnostic engine able to provide diagnosis of pet medical condition.

In one embodiment a conversation graph database can be used to store spoken inputs from persons and provide them to a veterinary diagnostic engine able to provide diagnosis of pet medical condition.

In one embodiment voice interface hardware can be provided for communication between a user and the veterinarian service manager.

In one embodiment a language processing module can be used for identifying critical key words and asking clarifying questions to provide them to a veterinary diagnostic engine able to provide diagnosis of pet medical condition.

In another embodiment, a system for providing veterinary services includes a communication network that supports audio communications and is able to receive a request for veterinary services for a pet. An automated veterinarian service manager able to provide diagnosis of pet medical condition based at least in part on information received through the communication network is used to direct a user to a veterinarian based on diagnosis of pet condition.

### BRIEF DESCRIPTION OF THE DRAWINGS

The specific features, aspects and advantages of the present invention will become better understood with regard to the following description and accompanying drawings where:

FIG. 1 illustrates a system supporting pet diagnosis and veterinarian recommendation;

FIG. 2 illustrates in more detail a system supporting pet diagnosis and veterinarian recommendation;

FIG. 3 illustrates one embodiment of a flow chart illustrating user interaction with a system supporting pet diagnosis and veterinarian recommendation;

FIG. 4 is a block diagram of a machine able to support aspects of the disclosed method and system; and

FIG. 5 illustrates an embodiment of a triage flow chart.

### DETAILED DESCRIPTION

In the following disclosure, reference is made to the accompanying drawings, which form a part hereof, and in which is shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Implementations of the systems, devices, and methods disclosed herein may comprise or utilize a special purpose or general-purpose computer including computer hardware, such as, for example, one or more processors and system memory, as discussed herein. Implementations within the scope of the present disclosure may also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

An implementation of the devices, systems, and methods disclosed herein may communicate over a computer network. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links, which can be used to carry desired program code means in the form of computer-executable instructions or data structures and which can be accessed by a general purpose or special purpose computer. Combinations of the above should also be included within the scope of computer-readable media.

Computer-executable instructions comprise, for example, instructions and data which, when executed at a processor, cause a general purpose computer, special purpose computer, or special purpose processing device to perform a certain function or group of functions. The computer executable instructions may be, for example, binaries, intermediate format instructions such as assembly language, or even source code. Although the subject matter is described in language specific to structural features and/or methodological acts, it is to be understood that the subject matter defined in the appended claims is not necessarily limited to the described features or acts described herein. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the description and claims to refer to particular system components. As one skilled in the art will appreciate, components may be referred to by different names. This document does not intend to distinguish between components that differ in name, but not function.

It should be noted that the sensor embodiments discussed herein may comprise computer hardware, software, firmware, or any combination thereof to perform at least a portion of their functions. For example, a sensor may include computer code configured to be executed in one or more processors and may include hardware logic/electrical circuitry controlled by the computer code. These example devices are provided herein for purposes of illustration and are not intended to be limiting. Embodiments of the present disclosure may be implemented in further types of devices, as would be known to persons skilled in the relevant art(s).

At least some embodiments of the disclosure are directed to computer program products comprising such logic (e.g., in the form of software) stored on any computer useable medium. Such software, when executed in one or more data processing devices, causes a device to operate as described herein.

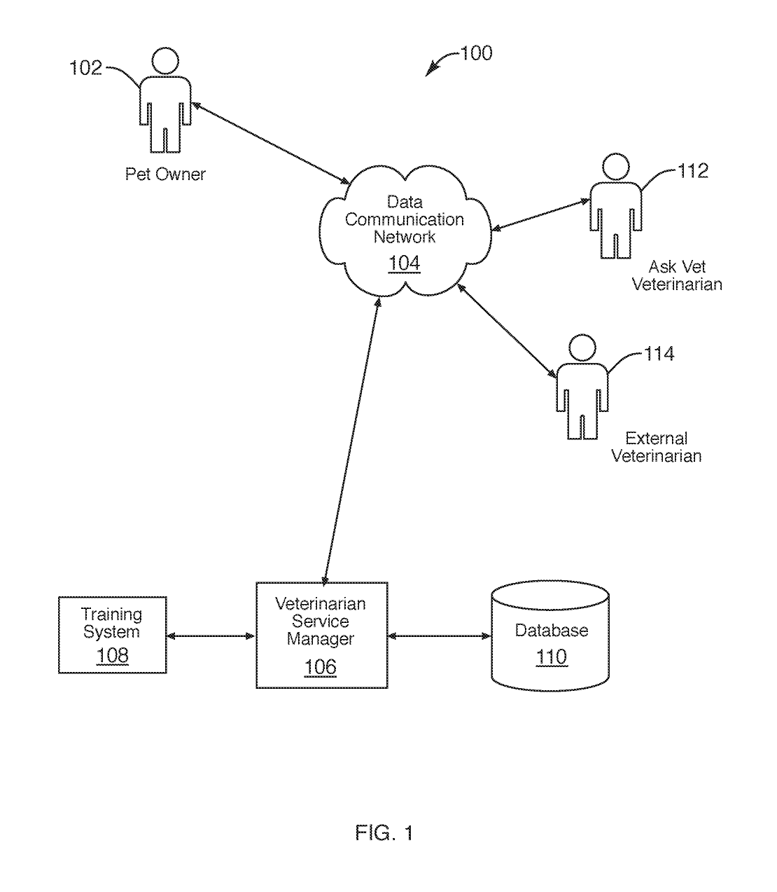

FIG. 1 is a block diagram depicting an environment 100 within which an example embodiment may be implemented. A pet owner 102 may communicate with a veterinarian service manager 106, a veterinarian 112, or a veterinarian 114 via one or more devices (not shown) coupled to a data communication network 104. For example, pet owner 102 may contact veterinarian service manager 106 to get information about a pet's medical condition. The veterinarian service manager 106 may use an artificial intelligence-based system to determine a severity of the pet's medical condition. Depending on the severity of the condition, veterinarian service manager 106 may connect pet owner 102 with veterinarian 112 or 114, thereby allowing pet owner 102 to talk directly with veterinarian 112 or 114. In some embodiments, veterinarian 112 works for (or is contracted by) the same entity that operates veterinarian service manager 106. Additionally, veterinarian 114 is an external veterinarian who does not work directly for the entity that operates veterinarian service manager 106. For example, veterinarian 114 may be an “on call” veterinarian who is contacted when other veterinarians 112 are not available.

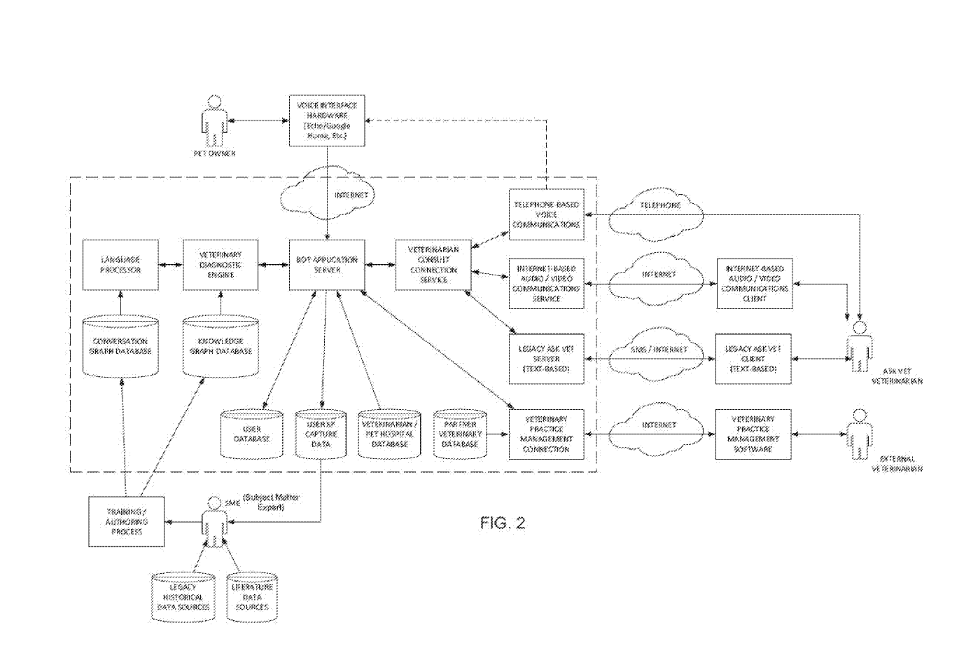

In some embodiments, data communication network 104 includes any type of network topology using any communication protocol. Additionally, data communication network 104 may include a combination of two or more communication networks. In some embodiments, data communication network 104 includes a cellular communication network, the Internet, a local area network, a wide area network, or any other communication network. As shown in FIG. 1, veterinarian service manager 106 is coupled to a training system 108 and a database 110. Training system 108 provides data related to animal-related medical conditions and treatments to veterinarian service manager 106. Similarly, database 110 may store various information related to animal-related medical conditions and treatments, as well as data related to pet owner 102, veterinarians 112, 114, and the like. FIG. 2 provides a more detailed explanation of the individuals, systems, devices, and components illustrated in environment 100.

FIG. 2 is a block diagram illustrating an embodiment of various components, systems, and devices, including both client devices and server devices, that allow a pet owner to interact with one or more of a veterinarian service manager (e.g., an automated system), a subject matter expert (SME), and a veterinarian. Many of the components, systems, and devices shown in FIG. 2 are described in greater detail below.

VOICE INTERFACE HARDWARE—network-connected computer system with a voice interface, such as Amazon Alexa, Google Home Assistant, Apple Siri, Microsoft Cortana, etc.

BOT APPLICATION SERVER—a service running on a network-connected computer system that is responsible for interfacing with multiple VOICE INTERFACE HARDWARE used by users over the internet. The BOT APPLICATION SERVER communicates with various other systems and servers depending on the state of the session to determine the appropriate response based on input from the VOICE INTERFACE HARDWARE.

USER DATABASE—a database containing information gathered about each user of the system, including such details as the user's name, phone number, pet types, pet names, previously diagnosed conditions, and all information collected about a user in the process of interacting with them or their individual pets. This information is provided to the VETERINARY DIAGNOSTIC ENGINE along with new data for each interaction with that user during a session.

USER XP CAPTURE DATA—a database that stores every interaction that every user has with the system by session for later analysis to improve the efficacy of the system.

VETERINARY DIAGNOSTIC ENGINE—a software system running on the same computer as the BOT APPLICATION SERVER, or on a separate server connected to it via a network, that drives the diagnostic portion of the application. For each session the VETERINARY DIAGNOSTIC ENGINE knows the context of that session including the previous answers provided during the dialog and other contextual information about the user, pet in question and other details from the USER DATABASE to determine either the next question to ask the user or deliver a conclusion (differential diagnosis, recommendations or referral) based on the conversation data stored in the CONVERSATION GRAPH DATABASE.

KNOWLEDGE GRAPH DATABASE—stores the knowledge base of diagnostic veterinary knowledge created by the subject matter experts that powers the automated differential diagnostic process. This data consists of term entities and conditions that is used to guide the system from one question to another and eventually to a conclusion based on the inputs received from the user during the session or previously.

LANGUAGE PROCESSOR—handles ambiguous inputs in order to determine the discrete answer to a voice query based on the KEYWORD DATABASE. The input for the LANGUAGE PROCESSOR is the open input from the user, with the output being the specific term entity that input likely corresponds to in the CONVERSATION GRAPH DATABASE. For example, the input might be “Poop” with the output being a known entity “Excrement” in the CONVERSATION GRAPH DATABASE.

CONVERSATION GRAPH DATABASE—a store of the words and phrases used by real people to describe specific known term entities as defined in the CONVERSATION GRAPH DATABASE.

VETERINARIAN/PET HOSPITAL DATABASE—a data store, possibly external third-party, of know veterinarians and pet hospitals used by the BOT APPLICATION SERVER when the user needs to be referred to an actual veterinarian or pet hospital near them. This database includes both facilities associated with and not associated with ASK.VET.

VETERINARIAN CONSULT CONNECTION SERVICE—a gateway service used by the BOT APPLICATION SERVER to connect the user to veterinarian via different means. Note that which means are used for any given sessions connection may be based on factors such as the user's preferences, the veterinarian's preferences, system availability, etc.

TELEPHONE-BASED COMMUNICATIONS—this component of the VETERINARIAN CONSULT CONNECTION SERVICE establishes a normal telephone-line connection between the user and the veterinarian via various means including via the system itself or an external direct connection. With this connection the veterinarian is answering a phone to interface with the system.

INTERNET-BASED AUDIO/VIDEO COMMUNICATIONS SERVICE—the component of the VETERINARIAN CONSULT CONNECTION SERVICE establishes a computer network connection between the INTERNET-BASED AUDIO/VIDEO COMMUNICATIONS CLIENT used by the veterinarian and the user which may include transmission of either audio only or both audio and video (either one-way or both-ways).

INTERNET-BASED AUDIO/VIDEO COMMUNICATIONS CLIENT—a software system used by the veterinarian on their own device (smartphone, tablet, laptop, PC, etc.) to connect with the system for audio and/or video communication. This software may be proprietary to Ask.Vet, part of the Ask.Vet web interface or a third-party system such as Skype, Hangouts, etc.

LEGACY ASK.VET SERVER—this component of the VETERINARIAN CONSULT CONNECTION SERVICE uses text-based communications from the user (via Speech-to-Text) to input the user's words into the existing text-based Ask.Vet service. The veterinarian can then type text back as used in the existing system which is read to the user using Text-to-Speech technology.

LEGACY ASK.VET CLIENT—this software is the existing software used by the current text-based Ask.Vet system.

VETERINARY PRACTICE MANAGEMENT CONNECTION—a gateway service used by the BOT APPLICATION SERVER to connect to a partner veterinarian's VETERINARY PRACTICE MANAGEMENT SOFTWARE in order to look up availability for veterinary practices near the user and to book an appointment with a chosen practice. This connection may also be used by the BOT APPLICATION SERVER to retrieve details about the user if a correlating entry for that user is established in their system.

PARTNER VETERINARY DATABASE—a data store of veterinary practices that have partnered with Ask.Vet to allow the scheduling of appointments via the system. This data store includes location data of the practice as well as details used by the VETERINARY PRACTICE MANAGEMENT CONNECTION to interface with the VETERINARY PRACTICE MANAGEMENT SOFTWARE.

TRAINING/AUTHORING PROCESS—this process, or set of processes, are the means by which subject matter experts take all the data available to them to construct the KEYWORD DATABASE and CONVERSATION GRAPH DATABASE. This includes both manual and automated processes.

LEGACY HISTORICAL DATA SOURCES—this data include conversations captured from the existing Ask.Vet text-based system.

LITERATURE DATA SOURCES—this data includes such things as industry texts and structure data from existing third-party diagnostic graph data stores.

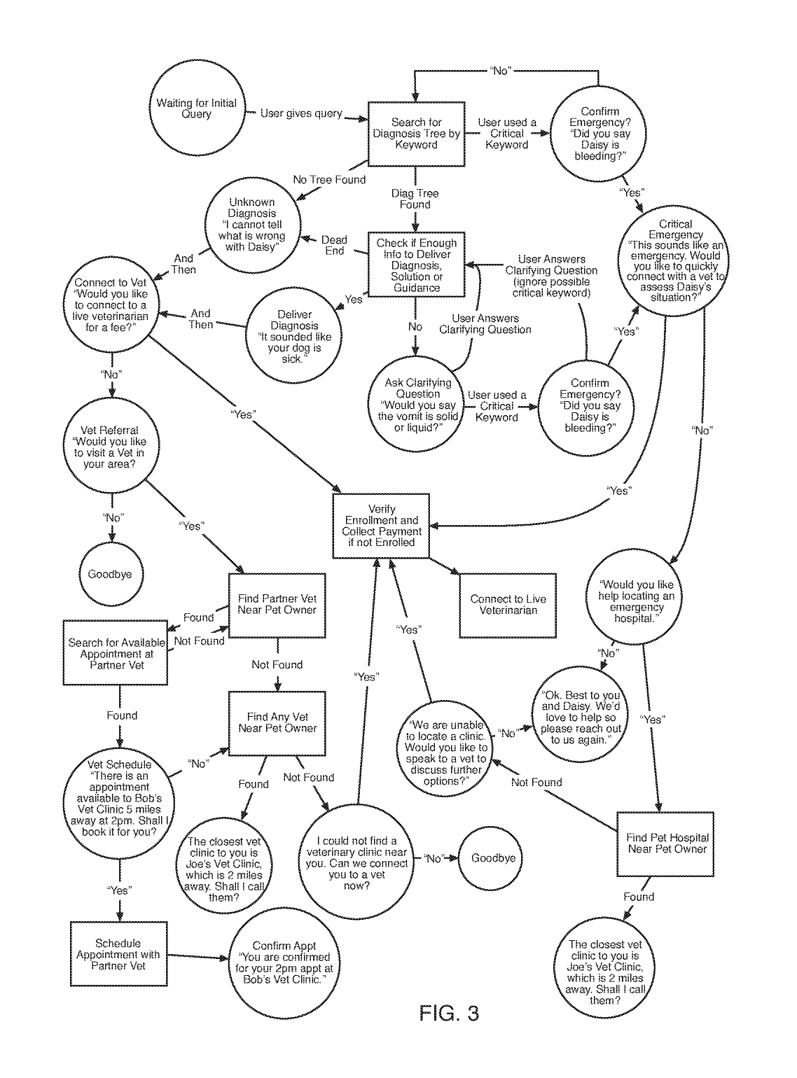

FIG. 3 is a flow diagram illustrating an example interaction and process flow between a user (e.g., a pet owner) and a veterinarian service manager via, for example, voice interface hardware. The example of FIG. 3 shows various questions and different process branches based on the answers to the questions, analysis of various data, search results for various information, and the like. Alternate embodiments may include different process flows, different process branches, different questions, different queries, different types of analysis, analyzing different types of data, and the like.

In one aspect of the illustrated embodiment, a user gives a query that is searched on a diagnosis tree through key word searching. If a diagnosis tree is found, a check is made to determine if enough information has been imparted to deliver a diagnosis, a solution, or guidance. If enough information has not been imparted, one or more clarifying questions can be asked. If a critical keyword is noted (e.g. bleeding or blood), an emergency can be confirmed and an option to connect with a veterinarian can be offered. If that offer to connect is accepted, user enrollment in the described system can be verified or offered, and payment made or verified before connection to a veterinarian is mediated.

In another aspect of the illustrated embodiment, a user gives a query that is searched on a diagnosis tree through key word searching. If a diagnosis tree is not found and no diagnosis is available, an offer to connect to a live veterinarian for fee can be made. If the user would like to visit a veterinarian near the user's location or area, a veterinarian can be found and an appointment scheduled. In some embodiments, distinctions can be made for veterinarians that are partner veterinarians who are connected and regularly work within the described system, and unaffiliated veterinarians.

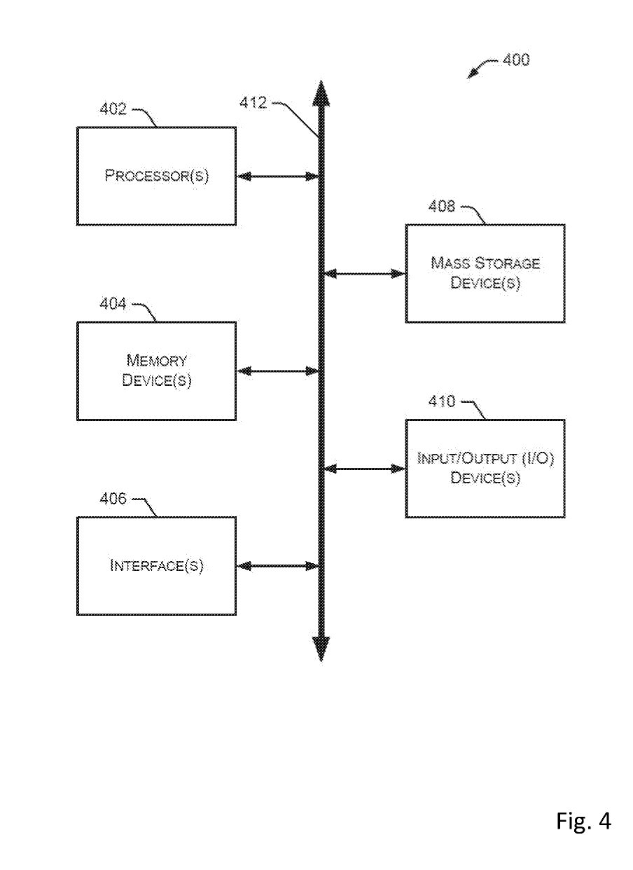

FIG. 4 is a block diagram of a machine in the example form of a computer system 400 within which a set of instructions, for causing the machine to perform any one or more of the methodologies discussed herein, may be executed. Computing system 400 may be used to perform various procedures, such as those discussed herein. Computing system 400 can function as a server, a client, or any other computing entity. Computing system 400 can be any of a wide variety of computing devices, such as a desktop computer, a notebook computer, a tablet computer, a server computer, a handheld computer, a smart phone, and the like.

Computing system 400 includes one or more processor(s) 402, one or more memory device(s) 404, one or more interface(s) 406, one or more mass storage device(s) 408, and one or more Input/Output (I/O) device(s) 410, all of which are coupled to a bus 412. Processor(s) 402 include one or more processors or controllers that execute instructions stored in memory device(s) 404 and/or mass storage device(s) 408. Processor(s) 402 may also include various types of computer-readable media, such as cache memory.

Memory device(s) 404 include various computer-readable media, such as volatile memory (e.g., random access memory (RAM)) and/or nonvolatile memory (e.g., read-only memory (ROM)). Memory device(s) 404 may also include rewritable ROM, such as Flash memory.

Mass storage device(s) 408 include various computer-readable media, such as magnetic tapes, magnetic disks, optical disks, solid state memory (e.g., Flash memory), and so forth. Various drives may also be included in mass storage device(s) 408 to enable reading from and/or writing to the various computer readable media. Mass storage device(s) 408 include removable media and/or non-removable media.

I/O device(s) 410 include various devices that allow data and/or other information to be input to or retrieved from computing system 400. Example I/O device(s) 410 include cursor control devices, keyboards, keypads, microphones, monitors or other display devices, speakers, printers, network interface cards, modems, lenses, CCDs or other image capture devices, and the like.

Interface(s) 406 include various interfaces that allow computing system 400 to interact with other systems, devices, or computing environments. Example interface(s) 406 include any number of different network interfaces, such as interfaces to local area networks (LANs), wide area networks (WANs), wireless networks, and the Internet.

Bus 412 allows processor(s) 402, memory device(s) 404, interface(s) 406, mass storage device(s) 408, and I/O device(s) 410 to communicate with one another, as well as other devices or components coupled to bus 412. Bus 412 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing system 400 and are executed by processor(s) 402. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

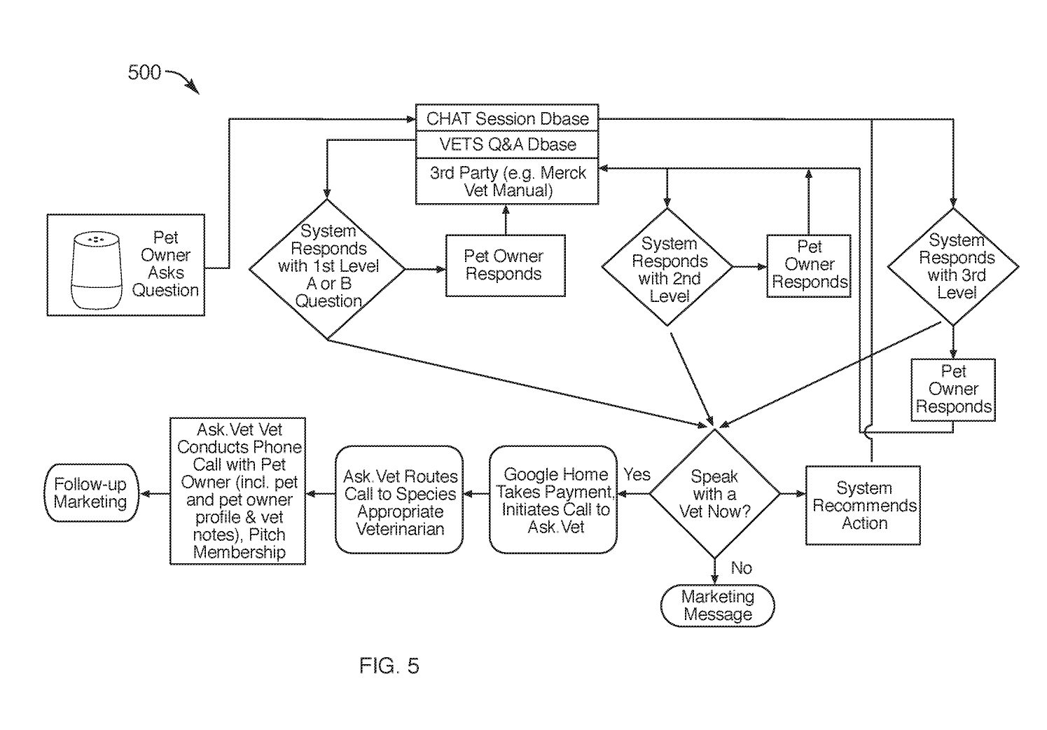

FIG. 5 illustrates an embodiment of a triage flow chart 500 illustrating user interaction via, for example, voice interface hardware.

While various embodiments of the present disclosure are described herein, it should be understood that they are presented by way of example only, and not limitation. It will be apparent to persons skilled in the relevant art that various changes in form and detail can be made therein without departing from the spirit and scope of the disclosure. Thus, the breadth and scope of the present disclosure should not be limited by any of the described exemplary embodiments. The description herein is presented for the purposes of illustration and description. It is not intended to be exhaustive or to limit the disclosure to the precise form disclosed. Many modifications and variations are possible in light of the disclosed teaching. Further, it should be noted that any or all of the alternate implementations discussed herein may be used in any combination desired to form additional hybrid implementations of the disclosure.

1. A method for providing veterinary services, the method comprising the steps of:
receiving a request for veterinary services for a pet through a communication network that supports audio communications;
providing diagnosis of pet medical condition based at least in part on information received through an automated veterinarian service manager; and
wherein the veterinarian service manager can direct a user to a veterinarian based on diagnosis of pet condition.

2. The method of claim 1, wherein the veterinarian service manager is connected to a training system able to provide data related to pet related medical conditions and treatments.

3. The method of claim 1, wherein the veterinarian service manager is connected to a database able to store and provide various information related to pet related medical conditions and treatments.

4. The method of claim 1, wherein the veterinarian service manager is connected to a database able to store and provide various information related to a pet owner, pet, or veterinarian.

5. The method of claim 1, wherein veterinarian service manager further comprises an artificial intelligence-based system to determine a severity of pet medical condition.

6. The method of claim 1, further comprising providing a knowledge graph database to store diagnostic veterinary knowledge and provide it to a veterinary diagnostic engine able to provide diagnosis of pet medical condition.

7. The method of claim 1, further comprising providing a language processor to handle ambiguous spoken inputs and provide them to a veterinary diagnostic engine able to provide diagnosis of pet medical condition.

8. The method of claim 1, further comprising providing a conversation graph database to store spoken inputs from persons and provide them to a veterinary diagnostic engine able to provide diagnosis of pet medical condition.

9. The method of claim 1, further comprising providing voice interface hardware for communication between a user and the veterinarian service manager.

10. The method of claim 1, further comprising identifying critical key words and asking clarifying questions to provide them to a veterinary diagnostic engine able to provide diagnosis of pet medical condition.

11. A system for providing veterinary services, comprising:
a communication network that supports audio communications and is able to receive a request for veterinary services for a pet;
an automated veterinarian service manager able to provide diagnosis of pet medical condition based at least in part on information received through the communication network; and
wherein the veterinarian service manager can direct a user to a veterinarian based on diagnosis of pet condition.

12. The system of claim 11, wherein the veterinarian service manager is connected to a training system able to provide data related to pet related medical conditions and treatments.

13. The system of claim 11, wherein the veterinarian service manager is connected to a database able to store and provide various information related to pet related medical conditions and treatments.

14. The system of claim 11, wherein the veterinarian service manager is connected to a database able to store and provide various information related to a pet owner, pet, or veterinarian.

15. The system of claim 11, wherein veterinarian service manager further comprises an artificial intelligence-based system to determine a severity of pet medical condition.

16. The system of claim 11, wherein the veterinarian service manager further comprises a knowledge graph database to store diagnostic veterinary knowledge and provide it to a veterinary diagnostic engine able to provide diagnosis of pet medical condition.

17. The system of claim 11, wherein the veterinarian service manager further comprises a language processor to handle ambiguous spoken inputs and provide them to a veterinary diagnostic engine able to provide diagnosis of pet medical condition.

18. The system of claim 11, wherein the veterinarian service manager further comprises a conversation graph database to store spoken inputs from persons and provide them to a veterinary diagnostic engine able to provide diagnosis of pet medical condition.

19. The system of claim 11, wherein the veterinarian service manager communicates with a user through voice interface hardware.

20. The system of claim 11, wherein the veterinarian service manager further comprises a language processing module for identifying critical key words and asking clarifying questions to provide them to a veterinary diagnostic engine able to provide diagnosis of pet medical condition.
